(19) United States
(12) Patent Application Publication
(10) Pub. No.: US 2008/0069897 A1
(43) Pub. Date: Mar. 20, 2008

(54) METHOD FOR PROMOTING AND MAINTAINING RESPIRATORY TRACT HEALTH

(76) Inventor: Tim Clarot, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/832,563

(22) Filed: Aug. 1, 2007

Related U.S. Application Data
(60) Provisional application No. 60/821,099, filed on Aug. 1, 2006.

(51) Int. Cl. A61K 33/20; A61K 33/00; A61K 36/00; A61K 36/534; A61K 36/886; A61P 43/00

(52) U.S. Cl. 424/606; 424/600; 424/680; 424/725; 424/744; 424/747

(57) ABSTRACT

A method for promoting and maintaining respiratory tract health by moisturizing and cleansing a nasal membrane is disclosed. The method includes the steps of applying a drug-free, preservative-free composition to an interior portion of a nasal cavity using a spray applicator. The spray applicator includes a microbiological filter to prevent the transfer of microbiological material between a nasal membrane and the composition.

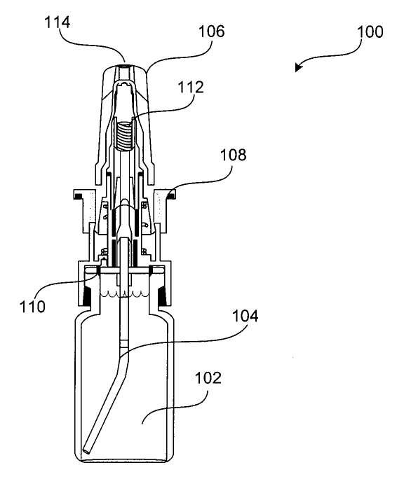

### FIELD OF THE INVENTION

The present invention generally relates to methods for promoting and maintaining health of a respiratory tract. More particularly, the invention relates to methods for cleansing and moisturizing nasal tissue.

### BACKGROUND OF THE INVENTION

The nasal cavity performs several functions that are important to the maintenance of the health of various animals. For example, in mammals such as humans, the nasal cavity warms, moistens, and cleanses air inspired into a respiratory tract and is considered to be the first line of defense against airborne disease and infection. Thus, it is generally desirable to maintain the health of the nasal cavity.

Most mammal nasal cavities include nasal hairs (vibrissae) at the opening of the nostrils to trap large particles of dust that might otherwise be inhaled, a nasal epithelium located at the lower portion of the nasal cavity to warm and moisten air and provide a chemical, mechanical and microbiological barrier to infection, and a mucous membrane to further filter the air. Generally, in order for the vibrissae, epithelium, and mucous membrane to work properly, each should remain relatively clean and moist.

Unfortunately, several environmental factors may cause a nasal cavity to become dirty and/or dry. For example, inhaled allergens or pollutants, such as dust, pollen, molds, animal dander, and grass may accumulate in the nasal cavity and may react with the mucous and result in a drying or irritation of the nasal cavity. Dry climates, cold climates, and changing seasons can also lead to nasal cavity dryness. In addition, colds, influenza, and other infections may cause an accumulation of material in the nasal cavity and dryness of the cavity, and typical pharmaceutical agents used to treat symptoms associated with such conditions may further cause the nasal cavity to become dry.

When the nasal cavity becomes dry or dirty, the cavity's ability to function properly becomes compromised. For example, as the cavity becomes dirty and/or dry, the cavity may become irritated, leading to general discomfort, and the cavity may be less able to cleanse and warm inspired air and/or ward off infection. Additionally, if left untreated, the dryness may lead to cracking and bleeding of the nose as well as various other acute or chronic conditions.

A common treatment for nasal membrane dryness includes use of a humidifier. Although the humidifier works well in many instances, the humidifier generally requires that the subject utilizing the humidifier remain proximate the humidified air for a period of time; such use may therefore be problematic or undesirable in some instances. Accordingly, improved methods for moisturizing the nasal membrane are desired. In addition, methods of cleaning the nasal cavity are desired to prevent unwanted dryness and promote and maintain respiratory health.

### SUMMARY OF THE INVENTION

The present invention provides an improved method for promoting and maintaining respiratory tract health. More particularly, the invention provides a method for promoting health by cleaning and moisturizing a nasal membrane. Cleaning and moisturizing a nasal membrane in accordance with the present invention prevents the onset of cold and allergy symptoms, reduces the severity of the symptoms after the onset of such symptoms, relieves pain associated with breathing, and promotes nasal comfort.

While the ways in which the present invention addresses the disadvantages of the prior art will be discussed in greater detail below, in general, the present invention promotes respiratory tract health by providing a convenient and effective method of cleaning and moisturizing a nasal membrane. The convenient, easy-to-use method of the invention promotes repeated and continued use of the method, which in turn additionally promotes respiratory tract health and comfort.

In accordance with one exemplary embodiment of the invention, a method for prophylaxis of cold or allergy symptoms includes providing an isotonic or hypertonic solution, providing an applicator designed to deliver a metered amount of the solution to a nasal membrane and to filter microbiological materials, such that the solution does not require any preservative or antimicrobial agent, and applying the solution to a portion of a nasal membrane to prevent the onset of cold or allergy symptoms. In accordance with various aspects of this embodiment of the invention, the composition is applied to a nasal cavity one or more times per day, even in the absence of any symptoms, to prevent the onset of cold or allergy symptoms. In accordance with various additional aspects of this embodiment, the solution includes one or more buffering agents, moisturizing agents, and/or essential oils.

In accordance with another embodiment of the invention, a method for reducing severity of symptoms associated with a cold or allergy includes providing an isotonic or hypertonic solution, providing an applicator designed to deliver a metered amount of the solution to a nasal membrane and to filter microbiological materials, such that the solution does not require any preservative or antimicrobial agent, and applying the solution to a portion of a nasal membrane to reduce a severity of symptoms associated with colds or allergies. In accordance with various aspects of this embodiment, the solution further includes one or more buffering agents, moisturizing agents, and/or essential oils. In accordance with yet further aspects of this embodiment, use of the method is continued, after any symptoms have subsided, to prevent the future onset and/or mitigate the severity of any additional cold or allergy symptoms.

In accordance with yet another embodiment of the invention, a method for reducing pain associated with breathing includes providing a drug-free, preservative-free solution, providing an applicator designed to deliver a metered amount of the solution to a nasal membrane and to filter microbiological materials, and applying the solution to a portion of a nasal membrane to reduce pain in the upper respiratory tract associated with breathing. In accordance with various aspects of this embodiment, the solution further includes one or more buffering agents, moisturizing agents, and/or essential oils.

In accordance with further embodiments of the invention, a method for promoting nasal comfort includes providing a composition including an isotonic or hypertonic solution, providing an applicator designed to deliver a metered amount of the solution to a nasal membrane and to filter microbiological materials, such that the solution does not require any preservative or antimicrobial agent, and applying the solution to a portion of a nasal membrane to increase nasal comfort. In accordance with various aspects of this embodiment, the solution further includes one or more buffering agents, moisturizing agents, and/or essential oils. In accordance with further aspects, the method promotes nasal comfort in dry and/or polluted environments.

In accordance with yet a further embodiment of the invention, a method for maintaining respiratory tract health includes providing a composition including an isotonic or hypertonic solution, providing an applicator designed to deliver a metered amount of the solution to a nasal membrane and to filter microbiological materials, such that the solution does not require any preservative or antimicrobial agent, and applying the solution to a portion of a nasal membrane to maintain respiratory tract health. In accordance with various aspects of the embodiment, the solution further includes one or more buffering agents, moisturizing agents, and/or essential oils.

### BRIEF DESCRIPTION OF THE DRAWING

The drawing figure illustrates a cut-away view of a spray applicator for use in accordance with various embodiments of the invention.

### DETAILED DESCRIPTION OF EXEMPLARY EMBODIMENTS

The present invention provides an improved method for maintaining and promoting respiratory tract health. As described in more detail below, the invention can be used for the prophylaxis and treatment of cold and allergy symptoms, for relieving pain associated with breathing, for promoting general nasal comfort, for maintaining general health of the respiratory tract, and the like.

The method includes applying a composition to the nasal membrane using a spray applicator. As described in more detail below, the compositions for use in accordance with the invention do not include any active ingredients. Furthermore, the applicator is designed to filter microbial agents, such that, even when the applicator is repeatedly inserted into a nasal cavity during use, antimicrobial agents and preservatives are not required in the composition. Because the compositions do not include active ingredients or antimicrobial agents, the compositions can be applied as frequently as desired, with no deleterious effect, and can therefore be used to promote and maintain respiratory tract health, even in the absence of any symptoms.

In accordance with various embodiments of the invention, the method is used periodically to treat specific symptoms, to prevent the onset of symptoms, or to provide general nasal comfort. However, as noted above, in addition to prophylaxis and treatment of various respiratory tract conditions through periodic use of the method, the method of the present invention encourages continued application of a composition and therefore provides additional promotion and maintenance of respiratory tract health—above that associated with the periodic use. In particular, the easy-to-use applicator in combination with a soothing, drug-free composition encourage continued use of the method and thus further promote respiratory tract health and comfort.

The drawing figure illustrates an easy-to-use, portable applicator 100 for use in accordance with various embodiments of the invention. Applicator 100 includes a reservoir 102 for storing a composition, an aspiration tube 104 for drawing the composition from reservoir 102, a tip 106 designed be inserted into a nostril to deliver the composition to a nasal membrane, a flange 108 to facilitate movement of tip 106 relative to reservoir 102, a molded, integrated microbiological filter 110 that allows ventilation for pump action and filters microbial material, a spring valve 112 to seal applicator 100 when not in use, and an opening 114.

In operation, a metered dose of the composition is applied to an interior portion of a nasal cavity by inserting tip 106 into a nostril and moving tip 106 relative to reservoir 102, in a pump action, to force the composition from reservoir 102 through tube 104 and out through tip 106. As tip 106 moves relative to reservoir 102, spring valve 112 opens a passageway between reservoir 102 and tip 106 to allow the composition to exit applicator 100. During operation, filter 110 prevents transfer of microbial agents from the nasal passage to the composition; thus, the composition does not require any preservatives or antimicrobial agents.

A dose administered in accordance with various embodiments of the invention may vary in accordance with several factors, such as intended use of the method, severity of a symptom and/or climate, and the like. By way of particular examples, apparatus 100 dispenses about 0.25 ml per pump and a dose is about 1 to about 4, and preferably about 1-2 pumps per nostril, administered 1-2 times per day. Any excess composition can be wipe away from the nasal cavity area.

The compositions for use with the present invention include isotonic or hypertonic solutions, including purified water and a salt, optionally buffering agents, and optionally an essential oil and/or a moisturizing agent. The compositions are sterile and do not include any preservative or drug, which might cause nasal irritation and/or contribute to dryness of a nasal membrane. Furthermore, as set forth in more detail below, some exemplary compositions are fragrance free and may therefore be used regularly by those with sensitivities to fragrances.

In accordance with various embodiments of the invention, a composition includes about 80-99 wt %, preferably about 90-98 wt %, and more preferably about 96-98 wt % purified water and about 1-5 wt %, preferably about 1.5-4 wt %, and more preferably about 2-3 wt % salt such as sodium chloride. The composition may also include about 0 or 0.001-2 wt %, preferably about 0.1-1.5 wt %, and more preferably about 0.5-1 wt % buffering agent. By way of particular examples, the composition includes about 0 or 0.001-1.5 wt %, preferably about 0.01-1.5 wt %, and more preferably about 0.1-1 wt % dibasic sodium phosphate and about 0 or 0.001-1 wt %, preferably about 0.01-0.5 wt %, and more preferably about 0.05-0.08 wt % potassium phosphate monobasic. The composition optionally includes an essential oil in an amount of about 0-0.1 wt %, preferably about 0.001-0.05 wt %, and more preferably about 0.001-0.2 wt % essential oil, such as spearmint oil. The composition further optionally includes a moisturizing agent in an amount of about 0-0.1 wt %, preferably about 0.001-0.05 wt %, and more preferably about 0.001-0.2 wt % moisturizing agent, such as aloe vera. All percents are weight percents of the total composition, unless otherwise indicated.

The examples provided below illustrate exemplary nasal moisturizing compounds for use with the method of the present invention. The examples are provided for illustration purposes only. The invention is not limited to the specific examples provided herein.

### EXAMPLE 1

| Component | Weight Percent |
|---|---|
| Purified Water | 96.68 |
| Sodium Chloride | 2.5 |
| Dibasic Sodium Phosphate | 0.75 |
| Potassium Phosphate Monobasic | 0.06 |
| Crystal, FCC | |
| Spearmint Oil | 0.01 |

In one study, the composition of Example 1 was administered to 115 women. A typical dose was 0.25 ml in each nostril. The results of the study showed that the composition was effective at cleaning and moisturizing a nasal membrane. In addition, the study revealed users enjoyed using the product, that the applicator and scented composition encouraged continued use of the method, and that application of the composition felt cool and soothing to participants in the study. Use of the composition relieved symptoms associated with colds, allergies, and sinusitis (e.g., cleared nasal passages of mucous and/or debris and relieved nasal dryness), relieved pain associated with breathing, made breathing easier, moistened mucous and facilitated removal of excess mucous, reduced occurrences of nose bleeds in arid environments, helped participants sleep and reduced snoring (when applying before sleep), reduced occurrence of allergy and cold symptoms such as sneezing and forming excess mucous, restored a sense of smell and taste after use of the product, and was nonirritating.

### EXAMPLE 2

| Component | Weight Percent |
|---|---|
| Purified Water | 96.68 |
| Sodium Chloride | 2.5 |
| Dibasic Sodium Phosphate | 0.75 |
| Potassium Phosphate Monobasic | 0.06 |
| Crystal, FCC | |
| Aloe Vera | 0.01 |

The composition of Example 2 was administered to subjects using the method of the present invention and similar results as those obtained with the composition of Example 1 were observed.

### EXAMPLE 3

| Component | Weight Percent |
|---|---|
| Purified Water | 97.49 |
| Sodium Chloride | 2.5 |
| Spearmint Oil | 0.01 |

The composition of Example 3 was administered to subjects using the method of the present invention and similar results as those obtained with the composition of Example 1 were observed.

### EXAMPLE 4

| Component | Weight Percent |
|---|---|
| Purified Water | 97.49 |
| Sodium Chloride | 2.5 |
| Aloe Vera | 0.01 |

The composition of Example 4 was administered to subjects using the method of the present invention and similar results as those obtained with the composition of Example 1 were observed.

As noted above, use of the method cleanses and moisturizes an interior portion of the nasal cavity, which promotes overall respiratory tract health and facilitates the proper function of the nasal cavity. Thus, use of the method may reduce an amount of or eliminate the need for any drug required to treat a respiratory tract disease or disorder or other disorder in which proper respiratory tract function promotes healing. For example, use of pharmaceutical decongestants, steroids, and the like may no longer be required to treat cold or allergy symptoms after use of the method of the invention. Alternatively, because the present method promotes nasal cavity and respiratory tract function, an amount of such pharmaceutical agents required to treat such a disease or disorder may be reduced.

The present invention has been described above with reference to exemplary embodiments. Those skilled in the art having read this disclosure will recognize that changes and modifications may be made to the embodiments without departing from the scope of the invention. For instance, the present invention has been described in connection with particular buffering agents and salts; however, various other buffers and salts may suitably be used with the method of the present invention. These and other changes or modifications are intended to be included within the scope of the present invention.

I claim:

1. A method for prophylaxis of cold or allergy symptoms, the method comprising the steps of:
providing a composition selected from the group consisting of isotonic solution and hypertonic solution;
providing an applicator configured to provide a metered amount of the composition to an interior portion of a nasal passage, wherein the applicator includes a mircofilter to prevent migration of microbial agents from an exterior portion of the applicator to the composition; and
applying the metered amount of the composition to the interior portion of the nasal passage to prevent the onset of cold or allergy symptoms.

2. The method for prophylaxis of cold or allergy symptoms of claim 1, wherein the step of providing a composition comprises providing a composition that is preservative free.

3. The method for prophylaxis of cold or allergy symptoms of claim 1, wherein the step of applying is performed in the absence of any cold or allergy symptoms.

4. The method for prophylaxis of cold or allergy symptoms of claim 1, wherein the step of providing a composition comprises providing a composition comprising a buffering agent.

5. The method for prophylaxis of cold or allergy symptoms of claim 1, wherein the step of providing a composition comprises providing a composition comprising a plurality of buffering agents.

6. The method for prophylaxis of cold or allergy symptoms of claim 1, wherein the step of providing a composition comprises providing a composition comprising an essential oil.

7. The method for prophylaxis of cold or allergy symptoms of claim 1, wherein the step of applying is performed after cold or allergy symptoms have subsided.

8. The method for prophylaxis of cold or allergy symptoms of claim 1, wherein the step of applying comprises spraying.

9. The method for prophylaxis of cold or allergy symptoms of claim 1, wherein the step of providing a composition comprises providing a composition comprising about 80 wt % to about 99 wt % purified water.

10. The method for prophylaxis of cold or allergy symptoms of claim 1, wherein the step of providing a composition comprises providing a composition comprising about 1 wt % to about 5 wt % salt.

11. The method for prophylaxis of cold or allergy symptoms of claim 1, wherein the step of providing a composition comprises providing a composition comprising about 1 wt % to about 5 wt % sodium chloride.

12. The method for prophylaxis of cold or allergy symptoms of claim 1, wherein the step of providing a composition comprises providing a composition comprising about 0.001 wt % to about 2 wt % buffering agent.

13. The method for prophylaxis of cold or allergy symptoms of claim 12, wherein the step of providing a composition comprises providing a composition comprising about 0.001 wt % to about 1.5 wt % dibasic sodium phosphate.

14. The method for prophylaxis of cold or allergy symptoms of claim 13, wherein the step of providing a composition comprises providing a composition comprising about 0.001 wt % to about 1 wt % potassium phosphate monobasic.

15. The method for prophylaxis of cold or allergy symptoms of claim 12, wherein the step of providing a composition comprises providing a composition comprising about 0.001 wt % to about 1 wt % potassium phosphate monobasic.

16. The method for prophylaxis of cold or allergy symptoms of claim 1, wherein the step of providing a composition comprises providing a composition comprising about 0 wt % to about 0.1 wt % essential oil.

17. The method for prophylaxis of cold or allergy symptoms of claim 1, wherein the step of providing a composition comprises providing a composition comprising about 0 wt % to about 0.1 wt % spearmint oil.

18. The method for prophylaxis of cold or allergy symptoms of claim 1, wherein the step of providing a composition comprises providing a composition comprising about 0 wt % to about 0.1 wt % moisturizing agent.

19. The method for prophylaxis of cold or allergy symptoms of claim 1, wherein the step of providing a composition comprises providing a composition comprising about 0 wt % to about 0.1 wt % aloe vera.

20. A method for preventing onset of cold or allergy symptoms, the method comprising the steps of:
providing a composition consisting essentially of about 80 wt % to about 99 wt % purified water, about 1 wt % to about 5 wt % salt, about 0 wt % to about 1.5 wt % of a first buffering agent, about 0 wt % to about 1 wt % of a second buffering agent, about 0 wt % to about 0.1 wt % moisturizing agent, and about 0 wt % to about 0.1 wt % of an essential oil;
providing an applicator configured to provide a metered amount of the composition to an interior portion of a nasal passage, wherein the applicator includes a mircofilter to prevent migration of microbial agents from an exterior portion of the applicator to the composition; and
applying the metered amount of the composition to the interior portion of the nasal passage to prevent the onset of cold or allergy symptoms.

21. A method for preventing onset of cold or allergy symptoms, the method comprising the steps of:
providing a composition cpnsosting essentially of about 80 wt % to about 99 wt % purified water, about 1 wt % to about 5 wt % sodium chloride, about 0 wt % to about 1.5 wt % dibasic sodium phosphate, about 0 wt % to about 1 wt % potassium phosphate monobasic, about 0 wt % to about 0.1 wt % aloe vera, and about 0 wt % to about 0.1 wt % of spearmint oil;
providing an applicator configured to provide a metered amount of the composition to an interior portion of a nasal passage, wherein the applicator includes a mircofilter to prevent migration of microbial agents from an exterior portion of the applicator to the composition; and
applying the metered amount of the composition to the interior portion of the nasal passage to prevent the onset of cold or allergy symptoms.
